(19) United States
(12) Patent Application Publication
(10) Pub. No.: US 2005/0048012 A1
(43) Pub. Date: Mar. 3, 2005

(54) Use of biotin or a biotin derivative for skin lightening purposes and for the treatment of senile lentigines

(76) Inventors: Roland Jermann, Laufen (CH); Helmut Luther, Grenzach-Whylen (DE)

Correspondence Address: Stephen M. Haracz;BRYAN CAVE LLP, 1290 Avenue of the Americas, New York, NY, 10104-3300, US

(21) Appl. No.: 10/833,983

(22) Filed: Apr. 28, 2004

(30) Foreign Application Priority Data
Aug. 26, 2003 (EP) 03018730.6

(51) Int. Cl. A61K 7/135

(52) U.S. Cl. 424/62; 514/393

(57) ABSTRACT

The present invention relates to the use of biotin and biotin derivatives alone, or with vitamin C or a derivative thereof, for the preparation of a cosmetic composition or of a pharmaceutical composition for skin-lightening purposes, for the elimination of skin color irregularities and for the treatment of senile lentigines.

### FIELD OF THE INVENTION

The present invention relates to the use of biotin or a biotin derivative alone, or with vitamin C or a vitamin C derivative, for the preparation of a pharmaceutical composition or a cosmetic composition for the treatment of senile lentigines, for smoothening skin color irregularities and/or for lightening natural skin color.

### BACKGROUND OF THE INVENTION

Senile lentigines are dark spots on the skin arising from the aging of the skin. They are a consequence of various aging processes, which are accelerated by incident light radiation. The skin thus appears inhomogeneous with respect to its color.

Tanning is a natural protective function of the skin with varying degrees of distinction in different ethnic groups. In many cultural circles a light skin tone is considered attractive so that a need for lightening the natural skin color arises.

Compositions for skin lightening purposes are known, such as e.g. hydroquinone, kojic acid, arbutin, vitamin C as well as various plant extracts. One problem with many compositions, however, is that apart from a lightening of the skin or an elimination of the liver spots also side effects such as e.g. skin irritations can occur. Plant extracts that lead to fewer skin irritations are generally not sufficiently effective.

There is a need for additional compositions, particularly cosmetic compositions that are well tolerated by the skin and yet are effective for skin lightening, for the treatment of senile lentigines and for smoothening skin color irregularities. The compositions should be at least as effective, preferably better than the familiar skin lightening compositions.

Biotin is a known active ingredient, which can be found in numerous cosmetic formulations and pharmaceutical compositions. Biotin is a compound of the following formula: 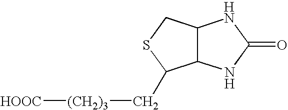
that can occur in eight different stereoisomeric forms. Biotin is in particular the D-(+)-biotin, i.e. the compound (3aS, 4S, 6aR)-2-oxohexahydrothieno[3,4-d]-imidazole-4-valeric acid of the following formula:

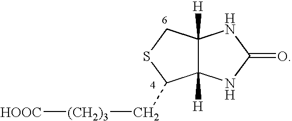

The effectiveness of the use of biotin in skin lightening applications has not been known until now.

### BRIEF DESCRIPTION OF THE FIGURES

FIG. 1 shows a bar graph comparing the differences in ITA° values over base line in groups of subjects administered a placebo and biotin (see Example 2). The ITA° values reflect the pigmentation of the skin and/or senile lentigines of the subjects.

FIG. 2 shows a bar graph comparing the differences in ITA° values over base line in groups of subjects administered a placebo and sodium ascorbyl phosphate (see Example 2). The ITA° values reflect the pigmentation of the skin and/or senile lentigines of the subjects.

FIG. 3 shows a bar graph comparing the differences in ITA° values over base line in groups of subjects administered a placebo and biotin+sodium ascorbyl phosphate (see Example 2). The ITA° values reflect the pigmentation of the skin and/or senile lentigines of the subjects.

### DETAILED DESCRIPTION OF THE INVENTION

Pursuant to the invention it was surprisingly found that biotin exhibits a skin-lightening effect and thus can be used for the treatment of liver spots, for smoothening skin color irregularities as well as for lightening the natural skin tone.

The invention hence makes the use of biotin available for the preparation of a composition for lightening the natural skin tone, for smoothening skin color irregularities and/or for treating senile lentigines.

The term “biotin” pursuant to the invention relates to the eight stereoisomers of the formula: 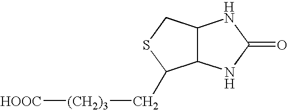
either in a stereochemically pure form or as any random mixture of two or more stereoisomers. Particularly preferred pursuant to the invention is the D-(+)-biotin of the formula:

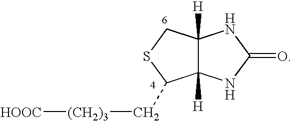

Biotin derivatives are known to the expert. These are compounds that are converted into biotin in vitro, particularly however in vivo. Lipophilic biotin derivatives, which generally penetrate the skin better than biotin itself, yet achieve equivalent effects as biotin, are particularly preferred. Apart from biotin, pursuant to the invention also biotin esters are particularly preferred, from which after penetration through the stratum corneum biotin is released again by the skin's own enzyme systems. Particularly preferred pursuant to the invention are biotin esters of the formulas I and II, which are deduced from biotin as follows: 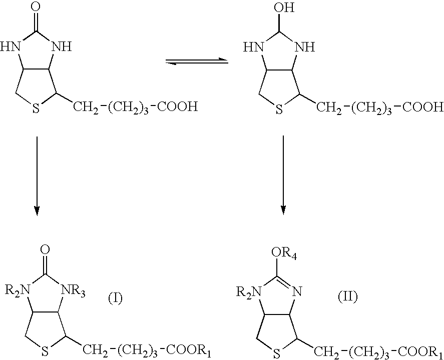
wherein
R₁=H, C₁-C₂₀-alkyl, C₅-C₇-cycloalkyl, aryl;
R₂ and R₃=independent from each other H, C₁-C₅-alkoxycarbonyl; and
R₄=H, C₁-C₂₀-alkyl, C₁-C₅-alkoxycarbonyl.

A C₁-C₂₀-alkyl radical is preferably a C₁-C₁₀-alkyl radical, even more preferred a C₁-C₆-alkyl radical such as a methyl, ethyl, n-propyl, iso-propyl, n-butyl, tert-butyl or an iso-butyl group.

A C₅-C₇-cycloalkyl radical is preferably a cyclohexyl group.

An aryl radical is preferably a C₅-C₁₀-aryl radical, in particular a phenyl group.

A C₁-C₅-alkoxycarbonyl radical is preferably a C₁-C₃-alkoxycarbonyl radical.

The radical R₁ is preferably a hydrogen atom or a C₁-C₆-alkyl radical.

At least one of the radicals R₂ or R₃ is preferably a hydrogen atom, even more preferred both radicals R₂ and R₃ are hydrogen atoms. The radical R₄ is preferably a hydrogen atom or a C₁-C₆-alkyl radical, even more preferred a hydrogen atom.

The radicals R₂, R₃ and R₄ are preferably all hydrogen atoms, and the radical R₁ is a C₁-C₆-alkyl radical, as defined above.

Among the biotin derivatives, all stereoisomeric forms and salts are included pursuant to the invention either alone or in random mixtures.

Pursuant to the invention, biotin and the derivatives thereof can be used individually, however it is also possible to employ a mixture of biotin and one or more of its derivatives, for example biotin in a mixture with one or more biotin esters, as defined above. Likewise various biotin derivatives can be employed in mixtures with each other.

The preparation of biotin derivatives is known to the expert, and in this respect conventional standard methods of organic chemistry can be used, e.g. the esterification of biotin with the desired alcohol such as methanol or ethanol by splitting off water.

The biotin and the biotin derivatives can likewise be used in the salt form. Suitable biotin salts are not particularly limited, and in this context salts with alkalis, alkaline earths and other suitable metals, but also with ammonium and organic bases, especially sodium, potassium, calcium and magnesium salts, can be mentioned. Due to the nitrogen atoms, the biotin and especially also the biotin derivatives can exist also in the form of an acid addition salt upon reaction with a suitable acid, such as an inorganic or organic acid, especially a mineral acid, e.g. with HCl. The hydrochloride salt is particularly preferred. Preparation of the salts occurs in the familiar fashion e.g. through reaction of the biotin or a derivative thereof with the corresponding base (e.g. NaOH or KOH) or the corresponding acid (e.g. HCl).

To the extent that within this description a “composition” is mentioned without more detailed specification, it should be understood both as a cosmetic composition and a pharmaceutical composition. To differentiate between cosmetic compositions and pharmaceutical compositions please refer e.g. to Römpp, Chemical Encyclopedia, 10th Edition and the literature cited therein. Pursuant to the invention biotin and/or a biotin derivative is preferably used for the production of a cosmetic composition in which the biotin is formulated together with additives that are compatible with cosmetics. Pursuant to the invention it is, however, also possible to use biotin for the production of a pharmaceutical composition, wherein the biotin is formulated with additives that are compatible with drugs. To the extent that within the framework of this application no other explanations are provided, the additives that are mentioned are additives that are compatible with cosmetics as well as additives that are compatible with drugs.

Pursuant to the invention, the compositions into which biotin and/or biotin derivatives are formulated are preferably topical compositions, such as e.g. liquid or solid oil-in-water emulsions, water-in-oil emulsions, multiple emulsions, micro-emulsions, PIT emulsions, Pickering emulsions, hydrogels, alcoholic gels, lipo-gels, single- or multiple-phase solutions, foams, ointments, plasters, suspensions, powders, creams, cleansers, soaps and other conventional compositions, which can also be applied e.g. by means of sticks, masks or as sprays.

The topical compositions preferably contain one or more additives, such as, for example, carriers and/or auxiliary agents that are compatible with cosmetic and/or pharmaceutical compositions, as they are generally used in such preparations. Here for example fats, oils, waxes, silicones, emulsifiers, alcohols, polyhydric alcohols, thickening agents, moistening and/or moisture-retaining substances, surfactants, softening agents, foam-retarding agents, anionic, cationic, non-ionic or amphoteric polymers, alkanization or acidification agents, water softeners, adsorbents, sun-screen agents, electrolytes, sequestering agents, water, organic solvents, preservatives, bactericides, anti-oxidants, vitamins, scents, aromas, sweetening agents, colorants and pigments can be mentioned.

The topical formulations pursuant to the invention preferably contain one or more conventional fatty substances as additives, e.g., vegetable oils, liquid paraffin oils, isoparaffin oils, synthetic hydrocarbons, di-n-alkyl esters, fatty acids, fatty alcohols, ester oils, hydroxy-carboxylic acid esters, di-carboxylic acid esters, diol esters, symmetrical, non-symmetrical or cyclic esters or carbonic acid esters with fatty alcohols, mono-, di- and tri-fatty acid esters with glycerin, waxes and silicon compounds.

The fatty substances are generally present in the topical composition in a quantity of 0.1 to 50% by weight, preferably from 0.1 to 20% by weight, in particular from 0.1 to 15% by weight (in relation to the entire composition, respectively).

The topical compositions can contain other additives, such as, for example, one or more surface-active substances as emulsifying or dispersing agents. Suitable examples of such emulsifying or dispersing agents are known.

The emulsifying agents can be present in the topical compositions for example in parts from 0.1 to 25% by weight, preferred from 0.5 to 15% by weight, in relation to the entire composition.

The topical compositions can likewise contain conventional sun-screen agents as additives, for example conventional UV-A and/or UV-B filters. An overview of conventional UV-A and UV-B filters, which can also be employed in the compositions pursuant to the invention, can be found for example in EP-A 1 081 140. Pursuant to the invention of course also novel sun protection filters that are disclosed in this document for the first time can be used in the inventive compositions.

Suitable organic, mineral or modified mineral sun-screen filters are also disclosed in WO 01/64177, to which we refer here as well.

If desired, the inventive compositions can also contain protein hydrolyzates or derivatives thereof as well as suitable mono-, oligo- or poly-saccharides or their derivatives, as additives, as they are e.g. revealed in WO 01/64177. Further suitable additives include auxiliary agents, such as vitamins, pro-vitamins and vitamin precursors, allantoin, bisabolol, anti-oxidants, ceramides and pseudo-ceramides, triterpenes, monomer catechines, thickening agents, plant glycosides, structure-providing substances (structuring agents), dimethylisosorbide, solvents, swelling and penetration adjuvants, perfume oils, pigments and colorants for dying the composition, substances for adjusting the pH value, complexing agents, opacifiers, pearly luster substances, expanding agents, film-forming, emulsion-stabilizing, thickening or adhesive polymers, especially cationic, anionic as well as non-ionic polymers are likewise revealed in WO 01/64177, which is incorporated herein by reference.

The compositions are preferably formulated such that they are suitable for topical applications. Topical application occurs preferably at least once a day, e.g. two or three times a day. The treatment duration generally lasts at least two days until the desired effect has been achieved. The treatment duration can also take several weeks or months.

The quantity of the composition that is to be applied depends on the concentration of the active ingredient in the composition as well as the severity of the disease that is to be treated and/or the desired cosmetic success. In the case of a pharmaceutical usage generally the quantity of the active ingredient to be used per application is higher than in the case of a cosmetic use. An effective amount for the application depends on the condition of the skin, the person to be treated as well as the severity and type of the skin discoloration to be treated and other factors, which are known to the attending physician or cosmetician. For example application can occur such that a cream is applied to the skin. A cream is usually applied in a suitable quantity of 2 mg cream/cm² skin. The applied quantity however is not critical, and if no treatment success should be achieved with a certain quantity of the applied active ingredient then the applied amount can certainly be increased, for example by using topical formulations with higher concentrations.

Pursuant to the invention the active ingredient can be formulated as such or also in encapsulated form, for example in liposomal form. Liposomes are beneficially formed with lecithins without or with the addition of sterols or phytosterols. The encapsulation of the active ingredient can occur alone or together with other active ingredients.

The inventive composition contains a suitable quantity of 0.0001% by weight to approximately 50% by weight of biotin in relation to the total weight of composition. It is more preferred if biotin is present in a suitable quantity of 0.01% by weight to about 20% by weight, even more preferred in a suitable quantity of about 0.01% by weight to about 1% by weight, in particular in a suitable quantity of about 0.1% by weight in relation to the total weight of the composition.

With respect to the type and preparation of the topical compositions as well as the disclosure of exemplary additives, we would like to refer to relevant literature, e.g. to NOWAK G.A., Cosmetic Preparations—Volume 2, Cosmetic Preparations—Recipes, Starting Substances, Scientific Basis (Verlag für chem. Industrie H. Ziolkowsky KG, Augsburg, Germany).

It is likewise possible to formulate biotin as an oral composition, for example in form of pills, tablets, capsules, which e.g. contain a granule or pellet, as liquid oral formulations or as an additive to foods, which is known to the expert in principle. Suitable methods and additives, with which the orally administered compositions can be produced pursuant to the invention, are disclosed e.g. in the standard work “Remington: The Science and Practice of Pharmacy”, Lippincott, Williams and Wilking (Publisher) 2000, which is incorporated herein by reference.

Traditional excipients such as micro-crystalline cellulose, sodium citrate, calcium carbonate, disodium or dipotassium phosphate, sodium or potassium phosphate, glycine, agents to promote breakdown such as starch or alginic acid, binding agents such as polyvinyl-pyrrolidone, saccharose, gelatin or acacia gum, slip additives such as magnesium stearate, sodium lauryl sulfate or talcum can be used in tablet production as conventional additives for oral compositions, especially for tablets. If the composition is filled in gel capsules, conventional auxiliary agents for the production of granules are lactose or milk sugar as well as polyethylene glycols with a high molecular weight. Further additives for other oral formulations, and in particular for the formulation as additives to foodstuffs, are known to the expert, and we refer to the relevant literature, e.g. “Principles of Food Engineering” (Grundzüge der Lebensmitteltechnik), Horst-Dieter Tscheuschner (publisher), 2nd, newly revised Edition Hamburg: Behr's 1996.

In case of an oral composition, the content of the active ingredient (i.e. the biotin and/or biotin derivative) in the composition is generally 1% to 90%, preferably 10% to 80%, e.g. 50% or more. Administration occurs such that the desired effect is achieved and depends on the condition of the patient, the type and severity of the skin discoloration to be treated, etc. and can easily be determined by the expert. A common daily dosage of the active ingredient is in the range from 0.1 μg/day to 50 mg/day, e.g. 20 μg/day to 2 mg/day.

Pursuant to the invention it was surprisingly found that apart from its own effectiveness for skin lightening purposes biotin exhibits a surprisingly high skin-lightening effect when it is administered together with vitamin C or a vitamin C derivative.

Vitamin C derivatives are known, and pursuant to the invention they are interpreted as all compounds that release vitamin C in vivo or in vitro, as well as solvates, hydrates and salts thereof. As examples of vitamin C derivatives e.g. glucosides of ascorbic acid and phosphates of ascorbic acid and in particular magnesium ascorbyl phosphate, sodium ascorbyl phosphate, calcium ascorbyl phosphate, potassium ascorbyl phosphate and mixed salts, such as e.g. sodium magnesium ascorbyl phosphate or sodium calcium ascorbyl phosphate, can be mentioned. Especially the phosphates frequently exist as hydrates, wherein the dihydrate form is the most common. Biotin is particularly preferred pursuant to the invention together with sodium ascorbyl phosphate, and the most preferred in form of the dihydrate, as it is available for example from Roche Vitamins AG under the product name STAY-C 50.

It has been known that vitamin C exhibits a skin-lightening effect, yet it was not known that a combination of biotin and/or biotin derivatives and vitamin C and/or a vitamin C derivatives have a skin-lightening effect that is considerably more distinct than the skin-lightening effect of vitamin C alone.

Pursuant to the invention the vitamin C and/or the derivatives thereof can be incorporated in the same formulation in which also the biotin and/or biotin derivative is present. Vitamin C and/or the derivative thereof in a topical composition is preferably used in a quantity of 0.001% by weight to about 50% by weight in relation to the total weight of the composition. It is more preferred if vitamin C and/or the derivative thereof is used in a topical composition in a quantity of 0.01% by weight to about 20% by weight, even more preferred in a quantity of about 0.1% by weight to about 15% by weight, e.g. 1 to about 5% by weight, such as e.g. 3% by weight, in relation to the overall weight of the composition. With respect to the quantity of vitamin C and/or the derivative thereof in an oral composition we would like to refer to the aforementioned embodiments of biotin and/or biotin derivatives, which also applies to the quantity and dosage of vitamin C and/or the derivative thereof.

Pursuant to the invention, the term “composition” also includes an embodiment in which the composition is present in two separate parts, wherein one part contains the active ingredient biotin or a derivative thereof and the other part the active ingredient vitamin C or a derivative thereof. The two separate parts of the composition can be topically applied, respectively, or orally ingested, respectively, yet it is also possible that one separate part of the composition is applied topically and the other part of the composition is administered orally so that in the inventive composition e.g. one separate part contains the active ingredient biotin and is applied topically, while the other separate part contains the active ingredient vitamin C or a derivative thereof and is administered orally or wherein the separate part of the composition that contains the active ingredient biotin is administered orally and the separate part of the composition that contains the active ingredient vitamin C and/or a derivative thereof is applied topically. The composition may be packaged in the form of a kit containing the biotin and vitamin C compounds in a single, uniform composition or packaged in separate containers that are administered in a number of different ways. Instructions for making and/or applying the composition are also part of the kit.

For the preparation of the separate parts of the composition, the additives, active ingredients and the quantities of the respective additives and active ingredients contained in the separate parts, reference can be made to the aforementioned examples of topical and oral formulations with biotin, which also apply to the inventive embodiment in which the composition exists in two separate parts, each containing an active substance. For the quantity of vitamin C and/or the derivative thereof in one of the separate parts, reference can be made to the aforementioned embodiments wherein the composition is not present in the form of two separate parts, but where both active ingredients are present in a single composition.

To the extent that the composition contains both biotin and/or a derivative thereof and vitamin C and/or a derivative thereof, the weight ratio of vitamin C and/or the derivative thereof to biotin and/or the derivative thereof is preferably 500:1 to 1:500, more preferred 100:1 to 1:100, and in particular 30:1 to 1:30. It is furthermore preferred that the quantity of vitamin C and/or the derivative thereof is higher in the composition than the quantity of biotin and/or the derivative thereof. The information above applies both to embodiments in which biotin and vitamin C and/or a derivative thereof are present together in the mixture and to embodiments in which the composition consists of two separate parts, wherein the one part contains the active ingredient biotin and/or a derivative thereof and the other part the active ingredient vitamin C and/or a derivative thereof.

To the extent that the active ingredients mentioned here can be present as hydrates or solvates, the hydrates and solvates are also included in the present invention.

Pursuant to the invention a composition that contains both active ingredients together in a mixture is preferred, particularly preferred is a composition that is administered topically.

The following examples are provided to further illustrate the process of the present invention. These examples are illustrative only and are not intended to limit the scope of the invention in any way.

### EXAMPLES

### Example 1

### Formulation Example

A cream was produced in the familiar fashion from the following components:

| | Ingredients | INCI Description | % w/w |
|---|---|---|---|
| A) | Brij 721 | Steareth 21 | 4.00 |
| | Brij 72 | Steareth 2 | 2.00 |
| | Lanette 0 | Cetearyl Alcohol | 2.00 |
| | Glyceryl Myristate | Glyceryl Myristate | 3.00 |
| | Oleic Acid | Oleic Acid | 6.00 |
| | Tegosoft M | Isopropyl Myristate | 3.00 |
| | Estol 1517 | Isopropyl Palmitate | 3.00 |
| | Transcutol CG | Ethoxydiglycol | 5.00 |
| | Phenonip | Phenoxyethanol & | 0.80 |
| | | Methylparaben & | |
| | | Ethylparaben & | |
| | | Propylparaben & | |
| | | Butyl para ben | |
| | Dow Corning 200, 350 cs | Dimethicone | 0.50 |
| | BHT | Butylated Hydroxytoluene | 0.05 |
| B) | Deionized water | Aqua | Ad 100 |
| | Propylene Glycol | Propylene Glycol | 5.00 |
| | Edeta BD | Disodium EDTA | 0.10 |
| | Keltrol T | Xanthan Gum | 0.20 |
| | Carbopol ETD 2001 | Carbomer | 0.30 |
| C) | TEA 99% | Triethanolamine | qs pH 7 |
| | Biotin | Biotin | 0.10 |
| | Deionized water | Aqua | 10.00 |
| D) | Deionized water | Aqua | 6.00 |
| | STAY-C 50 | Sodium Ascorbyl Phosphate | 3.00 |

Parts A) and B) were heated separately from each other to 75° C., respectively, while stirring. As soon as parts A) and B) were homogeneous, part B) was added to part A) while stirring. The mixture was homogenized at 11,000 RPM for 30 seconds. Part C) was pre-warmed to 65° C. and added to the homogenized mixture of A) and B). The mixture of A), B) and C) was cooled down to 40° C., and part D) was added. The mixture was cooled down to the ambient temperature (25° C.) while stirring.

The resulting cream had a pH value of 7.0 and had a viscosity (Brookfield RVT, 25° C., Spindle 5,10 RPM) of approximately 20,000 cP.

Apart from a cream pursuant to the invention, a placebo was produced correspondingly, in which neither sodium ascorbyl phosphate nor biotin were present, as well as a cream with 0.1% biotin exclusively and a cream with 3% sodium ascorbyl phosphate exclusively.

### Example 2

### Test Example

39 female subjects were divided into three groups of 13 persons each. The subjects applied twice a day for three months a test formulation on the left and a second test formulation on the right halves of their faces as well as on the left and the right backs of their hands. The test formulations were coded and corresponded to a placebo formulation and a formulation with the desired test substance. The three groups hereby tested the creams produced above with 3% sodium ascorbyl phosphate (STAY-C 50), 0.1% biotin and with a mixture of 3% sodium ascorbyl phosphate and 0.1% biotin.

A CR 300 chromometer was used to measure the lightening of the senile lentigines. The values that were obtained were provided as ITA° values. ITA° describes the pigmentation degree of the skin and/or the senile lentigines. The values reflected below correspond to the differences in ITA° values over the base line before start of the study. The higher the value, the greater the lightening of the skin. The ITA° values were determined after 29, 57 and 85 days, i.e. after approximately one month, after about two months and after about three months. The results are shown in the following table.

| | ITA° | | | ITA°-p-values | | |
|---|---|---|---|---|---|---|
| Composition | Day 29 | Day 57 | Day 85 | Day 29 | Day 57 | Day 85 |
| Placebo | 1.53 | 7.67 | 9.29 | 0.381 | 0.953 | 0.857 |
| 3% NAP | 4.46 | 7.53 | 8.80 | | | |
| Placebo | 2.75 | 5.93 | 8.07 | 0.156 | 0.480 | 0.217 |
| 0.1% Biotin | 5.57 | 7.55 | 11.16 | | | |
| Placebo | 3.32 | 6.89 | 9.70 | 0.055 | 0.006 | 0.045 |
| 3% NAP + | 7.16 | 11.65 | 13.42 | | | |
| 0.1% Biotin | | | | | | |

The study was conducted during the winter months, and during this time the skin lightens naturally. This explains why also the placebo formulations led to a slight skin lightening. The skin lightening effect for the placebo formulations however is only small.

Surprisingly biotin had a skin lightening effect already at a concentration of 0.1%, which is greater after one and three months and about as high as that of the known skin lightening composition vitamin C after two months. The very high skin lightening effect of a mixture of 3% sodium ascorbyl phosphate and 0.1% biotin was particularly surprising.

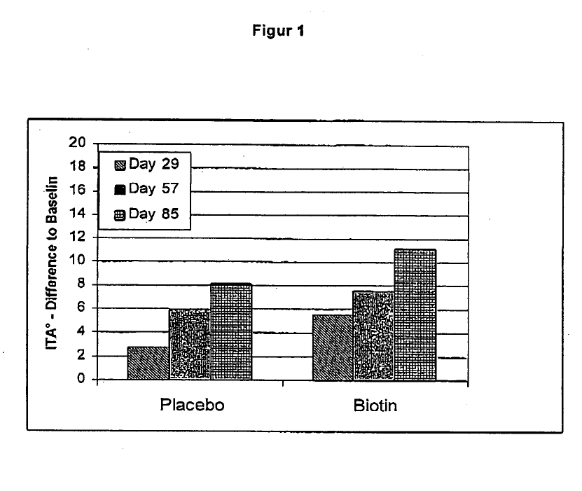

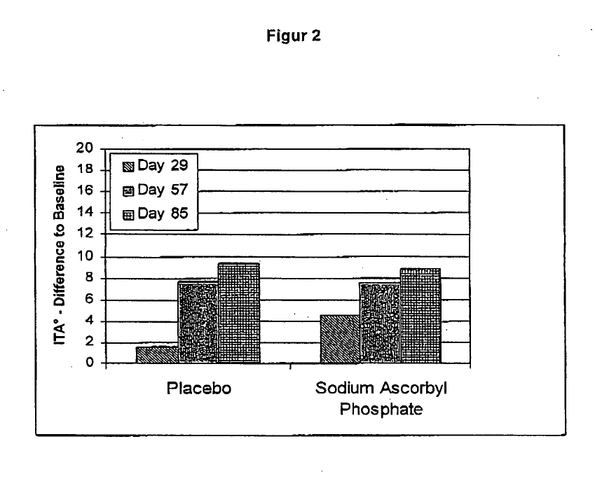

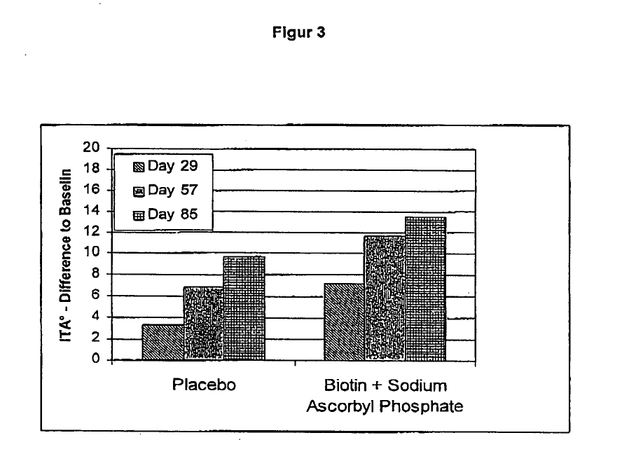

The results of the study are shown in FIGS. 1 through 3.

The invention being thus described, it will be obvious that the same may be varied in many ways. Such variations are not to be regarded as a departure from the spirit and scope of the invention and all such modifications are intended to be included within the scope of the following claims.

1. A method for lightening the skin, smoothening skin color irregularities and/or treating senile lentigines comprising administering to an individual in need thereof a composition comprising an effective amount of a biotin compound selected from the group consisting of biotin, biotin salts, biotin derivatives, biotin derivative salts, and mixtures thereof.

2. A method according to claim 1, wherein the biotin salt is selected from the group consisting of an alkali salt, an earth alkali salt, an ammonium salt, a hydrochloride, and mixtures thereof.

3. A method according to claim 1, wherein the biotin derivative salt is selected from the group consisting of an alkali salt, an earth alkali salt, an ammonium salt, a hydrochloride of a biotin derivative, and mixtures thereof.

4. A method according to claim 1, wherein the biotin derivative is a compound of formula I or formula II: 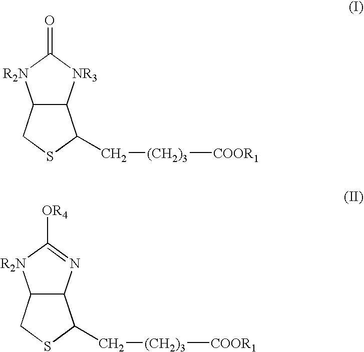
wherein
R₁ is H, C₁-C₂₀-alkyl, C₅-C₇-cycloalkyl, or aryl;
R₂ and R₃ are each independently H or C₁-C₅-alkoxycarbonyl; and
R₄ is H, C₁-C₂₀-alkyl or C₁-C₅-alkoxycarbonyl.

5. A method according to claim 1, wherein the composition is administered topically.

6. A method according to claim 1, wherein the composition is administered orally.

7. A method according to claim 1, wherein the composition is a cosmetic.

8. A method according to claim 1, wherein the composition is a pharmaceutical.

9. A method according to claim 1, wherein the biotin compound is present in the composition at a concentration of 0.001 to 50% by weight, in relation to the weight of the composition.

10. A method according to claim 1, wherein the biotin compound is present in the composition at a concentration of 0.01 to 1% by weight, in relation to the weight of the composition.

11. A method according to claim 1, wherein the composition further comprises a vitamin C compound selected from the group consisting of vitamin C, a vitamin C derivative and mixtures thereof.

12. A method according to claim 11, wherein the vitamin C derivative is sodium ascorbyl phosphate or a hydrate thereof.

13. A method according to claim 12, wherein the hydrate of sodium ascorbyl phosphate is a dihydrate.

14. A method according to claim 11, wherein the vitamin C compound is present in the composition at a concentration of 0.001 to 50% by weight, in relation to the weight of the composition.

15. A method according to claim 11, wherein the vitamin C compound is present at a concentration of 0.1 to 15% by weight, in relation to the weight of the composition.

16. A method according to claim 11, wherein the weight ratio of the vitamin C compound to the biotin compound is 500:1 to 1:500.

17. A method according to claim 11, wherein the weight ratio of the vitamin C compound to the biotin compound is 30:1 to 1:30.

18. A method according to claim 11, wherein the biotin compound is physically separated from the vitamin C compound.

19. A method according to claim 18, wherein one of the biotin and vitamin C compounds is administered orally and the other is administered topically.

20. A method for preparing a composition for lightening the skin, smoothening skin color irregularities and/or treating senile lentigines comprising:
combining a biotin compound selected from the group consisting of biotin, biotin salts, biotin derivatives, biotin derivative salts, and mixtures thereof with an additive.

21. A method according to claim 20, wherein the biotin derivative is a compound of formula I or formula II: 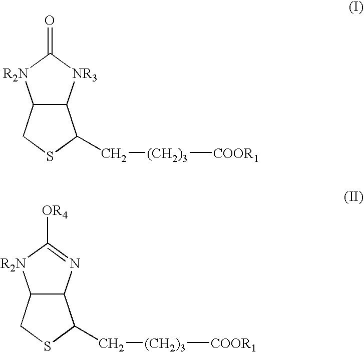
wherein
R₁, is H, C₁-C₂₀-alkyl, C₅-C₇-cycloalkyl, or aryl;
R₂ and R₃ are each independently H or C₁-C₅-alkoxycarbonyl; and
R₄ is H, C₁-C₂₀-alkyl or C₁-C₅-alkoxycarbonyl.

22. A method according to claim 20 further comprising:
combining a vitamin C compound selected from the group consisting of vitamin C, a vitamin C derivative and mixtures thereof with the biotin compound and the additive in any order.

23. A kit for lightening the skin, smoothening skin color irregularities and/or treating senile lentigines comprising:
(a) a composition comprising an effective amount of a biotin compound and optionally a vitamin C compound each, if present, stored together in a single formulation or separately in a distinct container, wherein the biotin compound is selected from the group consisting of biotin, biotin salts, biotin derivatives, biotin derivative salts, and mixtures thereof, and the vitamin C compound is selected from the group consisting of vitamin C, a vitamin C derivative and mixtures thereof; and
(b) instructions advising a consumer how to administer the composition.

24. A kit according to claim 23, wherein one of the biotin and vitamin C compounds is for oral administration and the other is for topical administration.

25. A kit according to claim 23, wherein the biotin and vitamin C compounds are present in a single composition, which is administered topically to skin.
